(19) United States
(12) Patent Application Publication
(10) Pub. No.: US 2009/0018512 A1
(43) Pub. Date: Jan. 15, 2009

(54) Pneumatically-Powered Ophthalmic Injector

(76) Inventor: Steven T. Charles, Memphis, TN (US)

Correspondence Address: ALCON, IP LEGAL, TB4-8, 6201 SOUTH FREEWAY, FORT WORTH, TX, 76134, US

(21) Appl. No.: 11/777,542

(22) Filed: Jul. 13, 2007

(51) Int. Cl. A61M 5/00

(52) U.S. Cl. 604/246; 604/117; 604/294

(57) ABSTRACT

An ophthalmic injection device has a dispensing chamber housing, a plunger, a shaft connected to the plunger, and a pneumatic cylinder. The dispensing chamber housing has an inner surface partially defining a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. The pneumatic cylinder has a piston movable in a chamber. The piston is connected to a shaft. The shaft is connected to the plunger. The shaft has teeth located on its surface. A pawl is engageable with the teeth and limits movement of the shaft to a dispensing direction. Pneumatic pressure introduced into the chamber moves the piston, shaft, and plunger in a dispensing direction.

### BACKGROUND OF THE INVENTION

The present invention relates to a single-use medical device and more particularly to a pneumatically-powered ophthalmic injection device for injecting a precise amount of a pharmaceutical, viscoelastic, perflurocarbon liquid, IOL, or the like.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

Syringes are also used during cataract surgery to place an intraocular lens into the eye. When age or disease causes the natural lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial lens.

The IOL is injected into the eye through the same small incision used to remove the diseased lens. The IOL is placed in an IOL injector in a folded state. The tip of the IOL injector is inserted into the incision, and the lens is delivered into the eye.

It would be desirable to have a portable hand piece for reliably injecting a pharmaceutical, viscoelastic, perfluorocarbon liquid, IOL, or the like. Since most surgical consoles have a source of pneumatic power, it would be desirable to have a disposable injection device that is easily connectable to the console and is pneumatically-powered.

### SUMMARY OF THE INVENTION

In one embodiment consistent with the principles of the present invention, the present invention is an ophthalmic injection device having a dispensing chamber housing, a plunger, a shaft connected to the plunger, and a pneumatic cylinder. The dispensing chamber housing has an inner surface partially defining a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. The pneumatic cylinder has a piston movable in a chamber. The piston is connected to a shaft. The shaft is connected to the plunger. Pneumatic pressure introduced into the chamber moves the piston, shaft, and plunger in a dispensing direction.

In another embodiment consistent with the principles of the present invention, the present invention is an ophthalmic injection device having a dispensing chamber housing, a plunger, a shaft connected to the plunger, and a pneumatic cylinder. The dispensing chamber housing has an inner surface partially defining a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. The pneumatic cylinder has a piston movable in a chamber. The piston is connected to a shaft. The shaft is connected to the plunger. The shaft has teeth located on its surface. A pawl is engageable with the teeth and limits movement of the shaft to a dispensing direction. Pneumatic pressure introduced into the chamber moves the piston, shaft, and plunger in a dispensing direction.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a cross section view of a pneumatically-driven ophthalmic injection device according to the principles of the present invention.

FIG. 2 is a cross section view of a pneumatically-driven IOL injection device according to the principles of the present invention.

FIG. 3A is a cross section view of a pneumatically-driven IOL injection device according to the principles of the present invention.

FIG. 3B is a cross section view of a pneumatically-driven IOL injection device according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

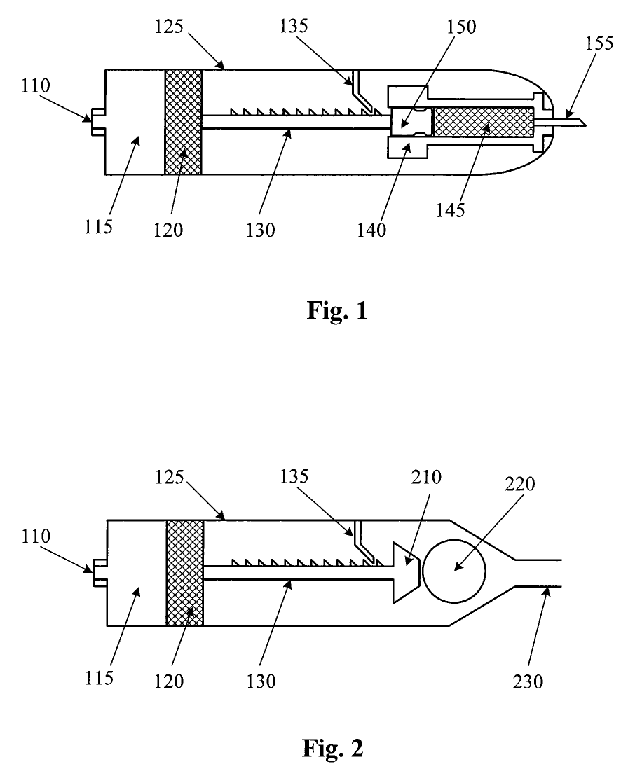

FIG. 1 is a cross section view of a pneumatically-driven ophthalmic injection device according to an embodiment of the present invention. In FIG. 1, the injection device includes a port 110, a chamber 115, a piston 120, a housing 125, a shaft 130, a pawl 135, a dispensing chamber housing 140, a dispensing chamber 145, a plunger 150, and a needle 155.

Port 110 is located on one end of the injection device, and needle 155 is located on the other end. A housing 125 encloses the various components depicted and forms an outer skin. Chamber 115 is fluidly coupled to port 110. Chamber 115 is configured to receive air (or a suitable gas or fluid) through port 110. Piston 120 is disposed in chamber 115 and forms one boundary of it. Piston 120 is capable of sliding in chamber 115 and is fluidly sealed to an inner surface of housing 125. In other words, piston 120 is fluidly sealed such that air introduced in chamber 115 pushes on piston 120 thus creating a driving force. One end of shaft 130 is attached to piston 120 such that movement of piston 120 results in a corresponding movement of shaft 130. The other end of shaft 130 is attached to plunger 150. Pawl 135 is located such that it engages teeth on shaft 130. Dispensing chamber housing 140 is configured to hold a substance to be delivered into the eye. One face of plunger 150 forms a boundary on one end of dispensing chamber 145. The interior surface of dispensing chamber housing 140 defines the rest of dispensing chamber 145. Needle 155 is fluidly coupled to dispensing chamber 145 such that a substance located in dispensing chamber 145 can be injected into an eye through needle 155.

Port 110 is designed to be coupled to a source of pneumatic power such as that found on the console of an ophthalmic surgical machine. Any other source of gas or fluid pressure may also be coupled to port 110. Such a gas or fluid is introduced into chamber 115 through port 110. Chamber 115 is adapted to receive the gas or fluid. Chamber 115 is of any suitable shape, and may be, for example, cylindrical in shape. In this case, the interior surface of housing 125 defines the shape of chamber 115.

Piston 120 is designed to fit in chamber 115 and create a fluid-tight seal with an interior surface of housing 125. Piston 120 is made of any suitable material and may contain sealing devices, such as o-rings. When a fluid, such as air, is introduced into chamber 115, a force is applied against piston 120. This force pushes piston 120 toward the needle end of the device. As is commonly known, piston 120 and chamber 115 may be implemented with a pneumatic cylinder. In other embodiments of the present invention, the piston and chamber mechanism may be implemented with a bellows mechanism, a diaphragm, a rolling edge diaphragm, a Bourdon actuator or other similar mechanism that is capable of converting pneumatic pulses into motion. Many such pneumatic mechanisms are commonly known.

Shaft 130 connects piston 120 to plunger 150. In this case, shaft 130 is made of a rigid material, such as a plastic. Teeth are disposed on one surface of shaft 130 as shown. These teeth engage pawl 135 to limit movement of shaft 130 to a single direction (toward the needle). In this case, as pressure is applied to a face of piston 120, piston 120 moves toward needle 155. Shaft 130 (connected to piston 120) also moves in the same direction. Pawl 135 slides over the teeth on shaft 130 as shaft 130 moves toward needle 155. When shaft 130 stops moving, pawl 135 prevents shaft from retracting (or moving in a direction opposite needle 155). In this manner, shaft 130 and connected plunger 150 are constrained to move in a single dispensing direction (toward needle 155). In other embodiments of the present invention, a ratchet and pawl mechanism may be employed. Other geared mechanisms may also be employed to limit motion of shaft 130 and plunger 150 to a single direction. The pawl and ratchet mechanism provides the same precision operation as a stepper motor with open loop control.

Dispensing chamber 145 contains a substance to be delivered into the eye. Dispensing chamber housing 140 and plunger 150 enclose dispensing chamber 145. Plunger 150 is fluidly sealed to an interior surface of dispensing chamber housing 140 to contain a substance located in dispensing chamber 145. Dispensing chamber 145 and dispensing chamber housing 140 may be of any convenient shape.

Needle 155 is fluidly coupled to dispensing chamber 145 and is adapted to deliver a substance, such as a pharmaceutical, viscoelastic, perfluorocarbon liquid, or the like, into an eye. Needle 155 may be of any commonly known configuration. Preferably, needle 155 is designed such that its characteristics are conducive to the particular delivery application. For example, when a pharmaceutical is to be delivered, needle 155 may be relatively short (several millimeters) in length to facilitate proper delivery of the pharmaceutical.

In operation, pneumatic pulses are introduced into chamber 115 through port 110. These pneumatic pulses produce a force that pushes piston 120, shaft 130, and plunger 150 toward needle 155. As plunger 150 slides in dispensing chamber 145, a substance contained therein is expelled through needle 155. Pawl 135 engages the teeth on shaft 130 such that its movement is only in a direction toward needle 155. In this case, once a substance is dispensed, plunger 155 cannot be retracted. Such a configuration prevents reflux and allows for precise delivery of a substance.

In addition, the number and size of the teeth on shaft 130 can be designed so that each tooth represents a small, precise movement of plunger 150 and a precise dosage of a substance from needle 155. In addition, the number and duration of the air pulses at port 110 can be controlled to control the quantity of the substance delivered and the rate of delivery of the substance through needle 155. The number of air pulses may be counted to determine the amount of substance injected (or the distance that the plunger moves).

A controller (not shown) functions to count the pneumatic pulses and/or monitor movement of the shaft. In this manner, the controller can precisely determine a dosage of the substance to be delivered into the eye. For example, the application of each pneumatic pulse may result in a corresponding amount of substance that is dispensed. The smaller the pneumatic pulses, the less substance is dispensed. Any gradation of dispensed substance can be achieved by precisely controlling the pneumatic pulses. Likewise, the controller may also be able to monitor the position of the shaft (or the distance the shaft travels). For example, the controller may be able to monitor the number of teeth that the pawl traverses. The controller may also monitor and direct the rate of movement of the piston.

The controller (not shown) is typically an integrated circuit with power, input, and output pins capable of performing logic functions. In various embodiments, the controller is a targeted device controller. In such a case, the controller performs specific control functions targeted to a specific device or component. In other embodiments, the controller is a microprocessor. In such a case, the controller is programmable so that it can function to control more than one component of the device. In other cases, the controller is not a programmable microprocessor, but instead is a special purpose controller configured to control different components that perform different functions.

FIG. 2 is a cross section view of a pneumatically-driven IOL injection device according to the principles of the present invention. In FIG. 2, an IOL 220 is located in the device. A plunger 210 pushes IOL 220 (which is typically in a folded state) through nozzle 230. IOL 220 is located in a compartment that is engaged with plunger 210. In this manner, movement of plunger 210 in the compartment results in movement of IOL 220 through nozzle 230. The operation of the device depicted in FIG. 2 is similar to the operation of the device depicted in FIG. 1.

In FIG. 2, the IOL injection device allows for precise movement of plunger 210 by the controlled application of pneumatic pulses at port 110. In addition, the size and configuration of the teeth on shaft 130 allow for precise movement of plunger 210 and a controlled delivery of the IOL 220.

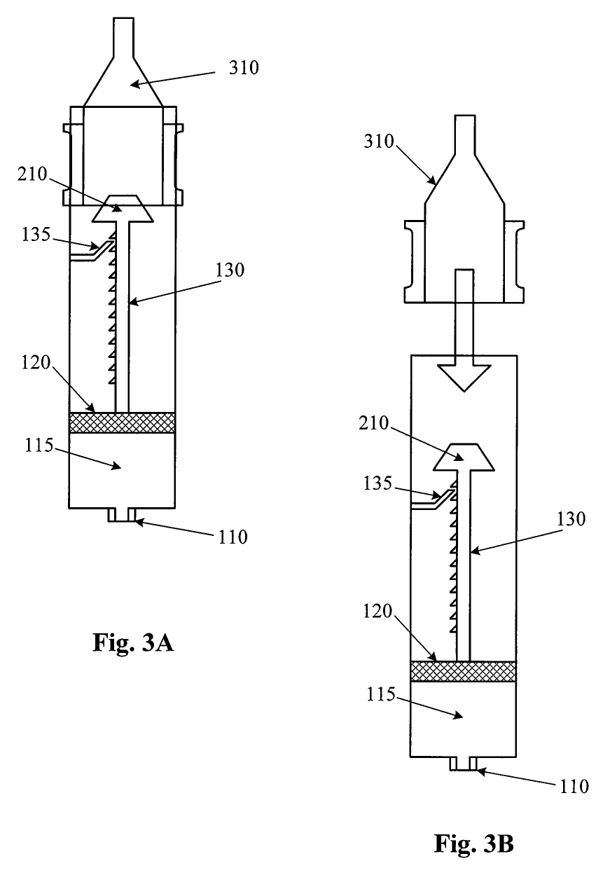

FIGS. 3A and 3B are cross section views of a pneumatically-driven IOL injection device according to the principles of the present invention. In FIGS. 3A and 3B, a cartridge 310 contains the IOL. Such a cartridge 310 may be placed into the injection device as shown. The removable cartridge 310 may be discarded after use. In other embodiments (such as those depicted in FIGS. 1 and 2), the entire device may be disposable.

From the above, it may be appreciated that the present invention provides an improved system for precisely delivering a pharmaceutical, viscoelastic, perflurocarbon liquid, IOL, or the like. The present invention provides a disposable, pneumatically-powered injection device. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic injection device comprising:
a dispensing chamber housing having an inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing;
a shaft connected to the plunger; and
a pneumatic mechanism connected to the shaft;
wherein pneumatic pressure introduced into the pneumatic mechanism moves the shaft and plunger in a dispensing direction.

2. The device of claim 1 further comprising:
a shaft connected to the plunger;
a piston connected to the shaft;
a port fluidly coupled to the chamber; and
a source of pneumatic power coupled to the port.

3. The device of claim 1 further comprising:
a needle fluidly coupled to the dispensing chamber.

4. The device of claim 1 wherein the shaft further comprises teeth located on a surface.

5. The device of claim 4 further comprising:
a pawl engageable with the teeth, the pawl for limiting movement of the shaft to the dispensing direction.

6. The device of claim 1 further comprising:
a ratchet and pawl mechanism coupled to the shaft.

7. The device of claim 1 wherein the plunger is only movable in the dispensing direction.

8. The device of claim 2 wherein controlled pulses of air delivered to the port result in a controlled dosage of the substance being injected into an eye.

9. The device of claim 2 further comprising:
a controller for counting pneumatic pulses, each pneumatic pulse defining a fixed quantity of a substance delivered from the dispensing chamber.

10. The device of claim 5 wherein the pitch of the teeth are designed so that a precise dosage of the substance is dispensed each time the pawl traverses a tooth.

11. The device of claim 5 further comprising:
a controller for monitoring movement of the shaft, such movement defining a dosage of the substance.

12. The device of claim 2 further comprising:
a controller for monitoring the rate at which the substance is delivered.

13. An ophthalmic injection device comprising:
a dispensing chamber housing having an inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing;
a needle fluidly coupled to the dispensing chamber;
a shaft connected to the plunger, the shaft having teeth located on its surface;
a pawl engageable with the teeth, the pawl for limiting movement of the shaft to a dispensing direction;
a piston connected to the shaft;
a chamber, the piston moveable in the chamber; and
a port fluidly coupled to the chamber;
wherein pneumatic pressure introduced into the chamber moves the piston, shaft, and plunger in the dispensing direction.
